(19) United States
(12) Patent Application Publication
(10) Pub. No.: US 2004/0193056 A1
(43) Pub. Date: Sep. 30, 2004

(54) Echographic examination method using contrast media

(76) Inventors: Elena Biagi, Firenze (IT); Leonardo Masotti, Firenze (IT); Luca Breschi, Prato (IT); Marco Scabia, Firenze (IT); Simona Granchi, Arezzo (IT)

Correspondence Address: MCGLEW & TUTTLE, PC, 1 SCARBOROUGH STATION PLAZA, SCARBOROUGH, NY, 10510-0827, US

(21) Appl. No.: 10/809,758

(22) Filed: Mar. 25, 2004

(30) Foreign Application Priority Data
Mar. 26, 2003 (IT) FI2003A000077

(51) Int. Cl. A61B 8/14

(52) U.S. Cl. 600/458

(57) ABSTRACT

What is described is an echographic examination method, in which an echographic contrast medium or agent, injected into a blood vessel and comprising a plurality of microbubbles, is sent by means of the blood circulation to a part of a living body under investigation and said part is struck by an ultrasonic excitation signal at an excitation frequency (f₀), and in which the microbubbles struck by the ultrasonic excitation signal generate an echo signal at a frequency different from the excitation frequency, said signal being used to, generate an image. The excitation signal exerts a pressure of 30 kPa to 1 MPa on said microbubbles, the microbubbles emitting a stable signal at not less than one subharmonic of the excitation frequency, said stable signal being processed to generate images.

### TECHNICAL FIELD

The present invention relates to a new method and a new device for carrying out echographic examinations using a contrast medium consisting of microbubbles.

### PRIOR ART

A procedure making use of echographic means or agents has been developed comparatively recently for the purpose of obtaining echographic images of blood vessels or other organs in living creatures. Very briefly, the method is based on the injection of a suspension of microbubbles, or of a substance which generates microbubbles when struck by an ultrasonic wavefront, into the patent under examination.

In recent years, the use of contrast agents or contrast media in fields other than ultrasonic diagnosis has produced a significant improvement in the quality of the final image.

Many research teams have made considerable efforts to characterize contrast agents, with the aim of investigating the mechanisms of interaction with ultrasound. Observations of contrast agents under the optical microscope and the development of theoretical models have yielded useful results concerning the physical behavior of the microbubbles, such as the agglomeration and fragmentation of microbubbles, even if these results are only partially applicable to an improvement of the quality of the ultrasonic image. It can be stated that the results of the ultrasonic observation of the contrast agent in different conditions of sonication have been sufficient to produce fundamental criteria for the proposal of innovative ultrasonic imaging methods.

Microbubbles struck by an ultrasonic wavefront at a given excitation frequency respond by back-propagating an echo at a frequency different from the excitation frequency.

U.S. Pat. No. 4,718,433 describes an echographic imaging method for application in the medical field, which makes use of a contrast medium of this type. Improvements to this examination method are described in U.S. Pat. Nos. 6,443,899; 6,221,017; 6,064,628; 6,034,922; 5,678,553; 5,410,516; 5,526,816 and 6,371,914. The entire content of these patents is expressly incorporated by reference in the present description, of which it is an integral part.

In practical applications, it has been found that the contrast medium struck by an ultrasonic wave emits a stable echo signal at the first harmonic, in other words at a frequency twice the excitation frequency. Although their existence has been reported in the literature and particularly in the United States patents cited above, emissions at subharmonics have never proved to be stable and consequently they are not used in practical applications.

Initial stages of research used microbubbles generated in a liquid, but the results were of limited practical use because of their instability. More recently, contrast medium consisting of microbubbles surrounded with shells or membranes were developed, and these gave better results because of the stability of the emission of the echographic response signal. Contrast medium for application in echographic examination are described in the following U.S. Pat. Nos.: 6,485,705; 6,403,057; 6,333,021; 6,200,548; 6,187,288; 6,183,725; 6,139,818; 6,136,293; 6,123,922; 6,110,443; 5,961,956; 5,911,972; 5,908,610; 5,840,275; 5,827,504; 5,686,060; 5,658,551; 5,597,549; 5,578,292; 5,567,414; 5,556,610; 5,531,980; 5,445,813; 5,413,774, and in European patents 554,213, 474,833, 619,743 and in international publication WO-A-9409829. The content of these publications is incorporated in full in the present description by reference and forms an integral part of it.

### OBJECTS AND SUMMARY OF THE INVENTION

The object of the present invention is to provide an echographic examination method using a contrast medium which makes it possible to obtain particular results which cannot be obtained with the conventional methods.

Essentially, the invention provides an echographic examination method in which an echographic contrast medium or agent, injected into a blood vessel and comprising a plurality of microbubbles, is sent by means of the blood circulation to a part of a living body under investigation and said part is struck by an ultrasonic excitation signal at an excitation frequency, and in which the microbubbles struck by the ultrasonic excitation signal generate an echo signal at a frequency different from the excitation frequency, said signal being used to generate an image. Characteristically, according to the invention, the excitation signal exerts a pressure of 30 kPa to 1 MPa on said microbubbles, so that the microbubbles emit a stable signal at one subharmonic at least, as well as at the harmonics of the excitation frequency, said stable signal being processed to generate images. Preferably, the pressure exerted by the ultrasonic waves is in the range from 40 to 900 kPa and even more preferably from 60 to 500 kPa. In a preferred embodiment, the pressure is in the range from 60 to 200 kPa.

The contrast medium can be one including microbubbles or that produces microbubbles upon exposure to ultrasound waves.

According to an aspect of the invention, the contrast medium is injected in a blood vessel of a patient in need of an ultrasound imaging investigation and an ultrasound image is generated using the subharmonic echo signal.

In another aspect, the present invention relates to an echographic examination method, in which an echographic contrast medium or agent, injected into a blood vessel and comprising a plurality of microbubbles, is sent by means of the blood circulation to a part of a living body under investigation, and said part is struck by an ultrasonic excitation signal at an excitation frequency, and in which the microbubbles struck by the ultrasonic excitation signal generate an echo signal at a frequency different from the excitation frequency, said signal being used to generate an image. Characteristically, the excitation signal exerts a pressure on said microbubbles sufficient to cause their rupture, and an echographic signal containing a spectral distribution at the excitation frequency, at its subharmonics and at its ultraharmonics is generated during the rupture, said signal being filtered to extract the spectral content from it at least two of said ultraharmonics and subharmonics. In practice, the signal is preferably filtered to extract from it all the frequency peaks at one or more subharmonics, harmonics or ultraharmonics, and the set of these data is used for the reconstruction of echographic images or for the extraction of information on the tissues under examination.

Further advantageous characteristics of the method according to the invention are indicated in the attached dependent claims.

The invention also relates to an echographic apparatus provided with an echographic probe and suitable means for reconstructing the echographic images, this apparatus being programmed to generate echographic excitation signals of the type described above and to use the signal at the frequency of at least one subharmonic of the excitation frequency.

### BRIEF DESCRIPTION OF THE DRAWINGS

The invention will be more clearly understood with the aid of the description and the attached drawings, which show some diagrams and results obtained with the method according to the invention. More particularly,

FIG. 1 shows in successive instants of time the temporal variation and the spectral content of the echographic signal obtained from an air bubble in water struck by an ultrasonic excitation signal;

FIG. 2 shows the echographic signal obtained from a microbubble of a Sonovue® contrast medium produced by Bracco International BV, Netherlands, at different values of acoustic pressure;

FIG. 3 shows two emission spectra obtained with the same contrast medium at two different excitation frequencies; and

FIG. 4 shows a B-mode representation of a plastic tube containing a liquid and a contrast medium, at the fundamental frequency, in other words the excitation signal frequency, and the subharmonic respectively.

### DETAILED DESCRIPTION OF THE INVENTION

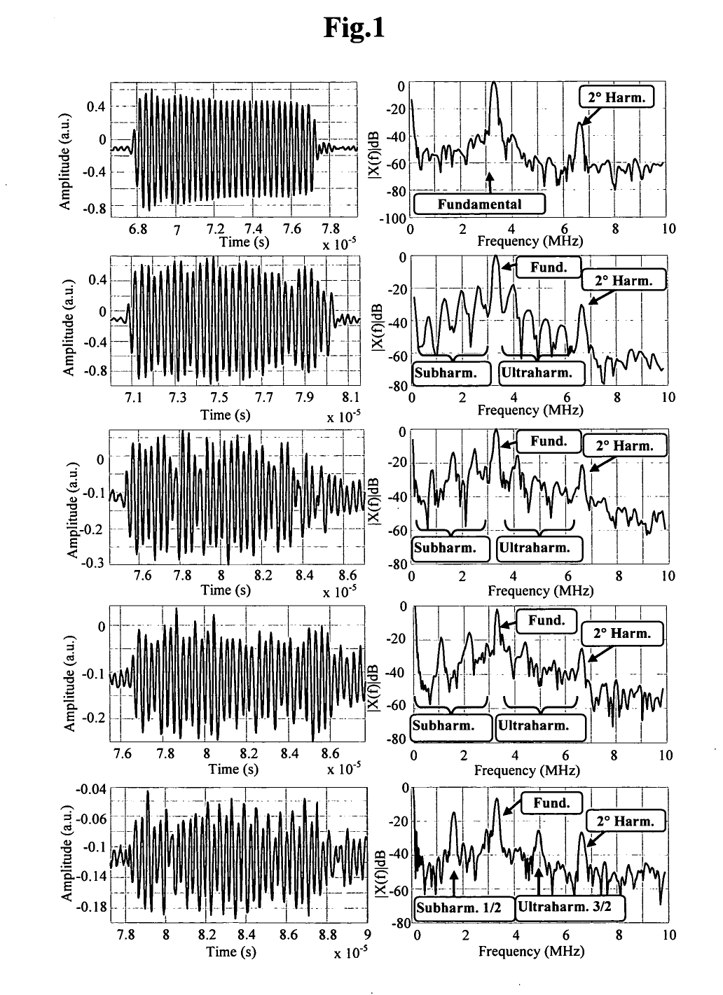

The diagrams in FIG. 1 show the behavior of a single air bubble during rupture. The air bubble under examination is produced by cavitation by injecting water through a needle with a fine aperture. This produces bubbles with diameters ranging from 10 to 100 μm. The experimental set-up consists of a radio frequency image acquisition platform, combined with the Esaote Megas echograph with a 3.3 MHz phased array echographic probe. In particular, the platform used is a “FEMMINA” platform, described in M. Scabia, E. Biagi, and L. Masotti, Hardware and software platform for real-time processing and visualization of echographic radiofrequency signals, in IEEE Trans. Ultrason. Ferroelect. Freq. Contr., 49, (2002), 1444-1452.

The bubbles are sonicated at high acoustic pressure (2 MPa-3 MPa) and the behavior of one particular bubble is observed.

FIG. 1 shows five successive instants of time in a temporal sequence having a total duration of 0.8 seconds, the radio frequency signals RF and their spectral distribution. At the acoustic pressure values which are used, the bubble is made to collapse or flash, emitting with a typical “comb-like” spectral content. In particular, subharmonics of different orders and ultraharmonic components appear in the destruction phase.

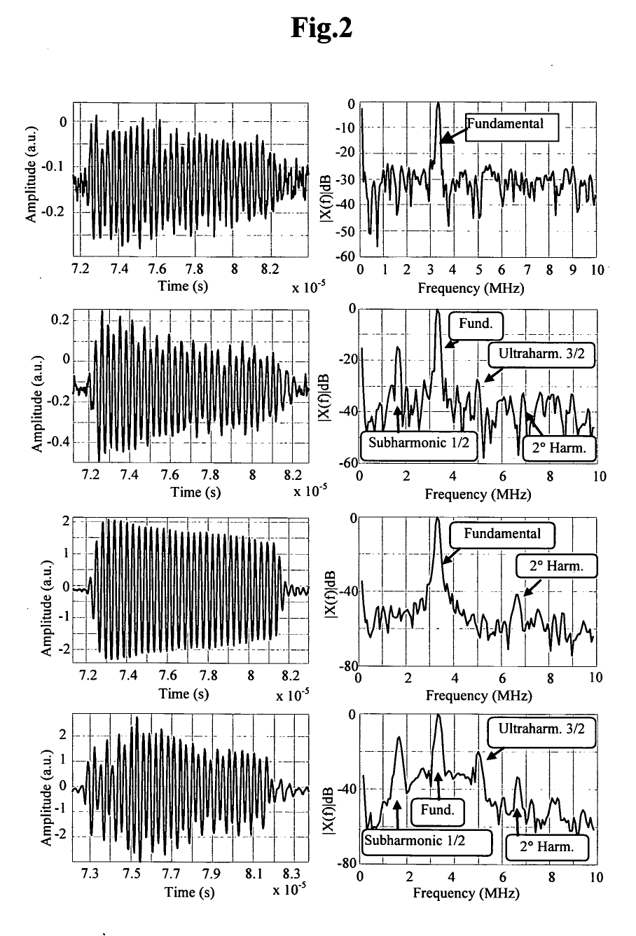

The following figure, FIG. 2, shows the result obtained with the same apparatus and a Sonovue® contrast medium or agent, again by analyzing the response of a single bubble. The Sonovue® contrast agent is available from Bracco International SA, the Netherlands, and is produced according to the teachings of patents EP-B-474833, EP-B-554213 and EP-B-619743.

The diagrams on the left in FIG. 2 show the temporal variation of the response signal, while the diagrams on the right show the variation of the frequency spectrum for different excitation conditions.

The excitation signal consists in all cases of an excitation pulse or burst consisting of thirty cycles at a frequency of 3.3 MHz (excitation frequency f₀). Reading downward, the four diagrams show the echographic response of the single bubble of contrast medium at different amplitudes of the excitation signal, in other words at different excitation pressures. In the first diagram, the excitation pressure is 35 kPa. As seen in the diagram on the right, the response contains no harmonics or subharmonics, but only a peak at the fundamental frequency of 3.3 MHz.

In the second case, the excitation pressure is 80 kPa. A stable emission is observed at the fundamental frequency and at the subharmonic ½ f₀.

When the amplitude of the excitation signal is increased further until the pressure is raised to 980 kPa, as shown in the third pair of diagrams, only the fundamental frequency f₀ and the harmonic 2 f₀ are present, while no back-propagation is seen at the subharmonics.

At sufficiently high acoustic pressures, the microbubbles are ruptured. This situation is seen in the fourth pair of diagrams, where the pressure is of the order of 1.5 MPa. When an excitation signal at this level is used, the destruction of the microbubble causes an emission of back-scattered ultrasound with a comb-like spectrum, in which a subharmonic at ½ f₀ and an ultraharmonic at 3/2 f₀ can be identified in addition to the fundamental frequency and the second harmonic.

Overall, the diagrams of FIG. 2 show that the Sonovue® microbubbles have stable subharmonic emissions at low pressure levels (80 kPa). When the bubble is sonicated with a high pressure level (980 kPa), the subharmonic spectrum disappears. It was found for the first time that the subharmonic emission is controlled by two pressure thresholds, the first being associated with its generation, while the second causes its disappearance, as shown in FIG. 2 where the RF signal back-propagated from the bubble is shown with its spectral distribution. The RF signal and its spectrum shown at the bottom of FIG. 2 refer to the destruction of the bubble and the subharmonic spectrum appears only at this moment for a very short interval.

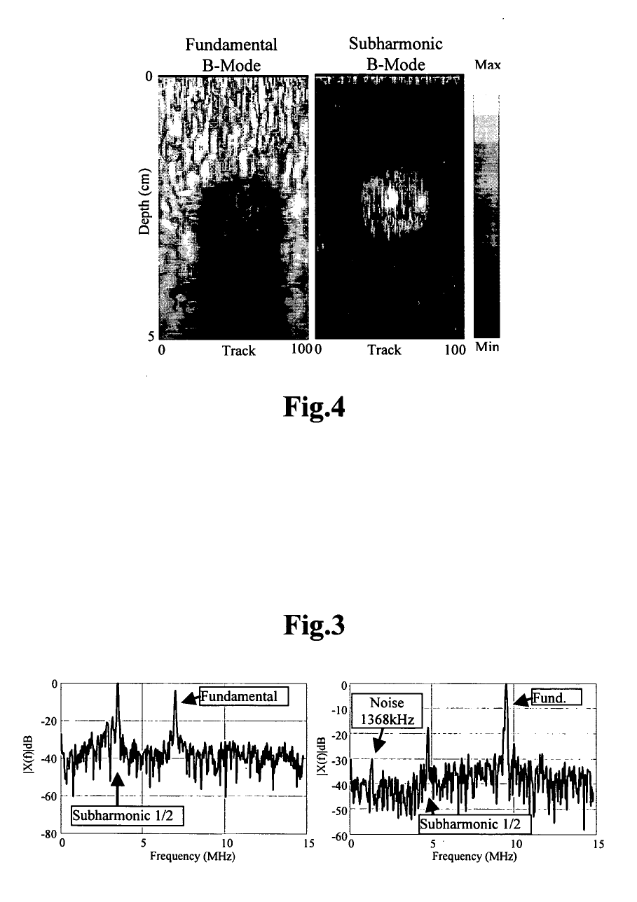

FIG. 3 shows the subharmonic stable emission spectra at low pressure and with an excitation pulse at two different central frequencies, 7 MHz and 9.5 MHz. 0.01 ml of Sonovue® dispersed in a liter of water was used for this measurement. Single-element transducers were used as the transmitter and a receiver with a Toellner TOE 7708° as a pulse generator. The receiving unit was a Panametrics 5052PR connected to the echographic acquisition platform for the acquisition and processing of the signals. The left-hand diagram in FIG. 3 shows the spectral distribution obtained by using a Gilardoni 5 MHz single-element transducer as the transmitting element and a Panametrics V382 3.5 MHz device as the receiving element.

The right-hand diagram in FIG. 3 shows the spectrum obtained with a Panametrics V311 10 MHz transmission transducer and a Gilardoni 5 MHz device as the receiving element.

The excitation signal used was a sinusoidal pulse or burst with a duration of 10 microseconds, containing 50 cycles, at a pressure of 70 kPa. In both diagrams, a response is seen at a frequency equal to the excitation frequency and at a frequency equal to the subharmonic ½ f₀.

By using the signal back-propagated from the contrast agent at the subharmonic of the excitation frequency, high-contrast images can be obtained.

The images shown in FIG. 4 were obtained by using an Esaote LA523 linear array with Esaote MEGAS front end hardware, connected to the RF image acquisition platform. The specimen consisted of a plastic tube filled with Sonovue® in water at a concentration of 0.05 ml per liter of water and immersed in an absorbent and diffusing fluid to simulate the attenuation of soft biological tissues. The subharmonic image shown on the right in FIG. 4 was obtained from a 91-tap Hanning filter centered on the subharmonic frequency. This image shows a very high contrast by comparison with the simulated tissue, since the signal back-scattered by the tube containing the fluid and by the surrounding absorbent fluid is completely eliminated.

This can be taken as a further confirmation that the subharmonic emission is a peculiar effect of the microbubble, whereas no subharmonic contribution is derived from the tissue simulator.

In conclusion, a full development of the microbubble up to and including its rupture and disappearance was shown in various measurement conditions. The simultaneous visualization of multiple images for different ultrasonic parameters made it possible to discover and emphasize certain specific effects in relation to the dynamics of interaction between microbubbles and ultrasound. It was found that the creation of the subharmonic was a phenomenon with an ultrasonic pressure threshold. In particular, it was demonstrated that even very low pressure levels activated the subharmonic emission.

The stability of the subharmonic emission at these low pressure levels was also observed.

Observation of the dynamics of a single bubble revealed the different behaviors of Sonovue® and the air bubbles, the latter showing a typical “comb-like” spectral fragmentation. As regards imaging methods using contrast agents, the most important result was the stability of the subharmonic emission and its occurrence at low pressure levels. Indeed, given that biological tissues do not show subharmonic emissions while they generate a second harmonic response, very useful future developments of signal processing methods can be expected.

1. Echographic examination method, in which an echographic contrast medium including microbubbles, or generating microbubbles upon exposure to ultrasonic waves, injected into a blood vessel, is sent by means of the blood circulation to a part of a living body under investigation and said part is struck by an ultrasonic excitation signal at an excitation frequency (f₀), and in which the microbubbles struck by the ultrasonic excitation signal generate an echo signal at a frequency different from the excitation frequency, said signal being used to generate an image, wherein said excitation signal exerts a pressure of 30 kPa to 1 MPa on said microbubbles, so that the microbubbles emit a stable signal at one subharmonic at least of the excitation frequency, said stable signal being processed to generate images.

2. Method according to claim 1, wherein said excitation signal exerts a pressure in the range from 40 to 900 kPa, preferably from 60 to 500 kPa, and even more preferably from 60 to 200 kPa on said microbubbles.

3. Method according to claim 1, wherein said excitation signal is a sinusoidal signal.

4. Method according to claim 1, wherein each of said microbubbles consists of a membrane containing a gaseous medium.

5. Method according to claim 2, wherein each of said microbubbles consists of a membrane containing a gaseous medium.

6. Method according to claim 3, wherein each of said microbubbles consists of a membrane containing a gaseous medium.

7. Method according to claim 1, wherein a plurality of images obtained at successive instants of time of the echographic signal, or at spatially distinct points of said part under examination, are displayed simultaneously on a screen.

8. Echographic examination method, in which an echographic contrast medium containing microbubbles or generating microbubbles upon exposure to ultrasonic waves, injected into a blood vessel, is sent by means of the blood circulation to a part of a living body under investigation and said part is struck by 5 an ultrasonic excitation signal at an excitation frequency (f₀), and in which the microbubbles struck by the ultrasonic excitation signal generate an echo signal at a frequency different from the excitation frequency, said signal being used to generate an image, wherein said excitation signal exerts sufficient pressure on said microbubbles to cause their rupture, an echographic signal being generated during the rupture said signal containing a spectral distribution at the excitation frequency, at its subharmonics and at its ultraharmonics, said signal being filtered to extract the spectral content from it at at least two of said ultraharmonics and subharmonics.

9. Method according to claim 8, wherein a plurality of images obtained at successive instants of time of the echographic signal or at spatially distinct points of said part under examination, are displayed simultaneously on a screen.

10. Ultrasonic method for imaging, in which an echographic contrast medium including microbubbles, or generating microbubbles upon exposure to ultrasonic waves, is introduced into a portion of a body under investigation and is struck by an ultrasonic excitation signal at an excitation frequency (f₀), and in which the microbubbles struck by the ultrasonic excitation signal generate an echo signal at a frequency different from the excitation frequency, said signal being used to generate an image, wherein said excitation signal exerts a pressure of 30 kPa to 1 MPa on said microbubbles, so that the microbubbles emit a stable signal at at least one subharmonic of the excitation frequency, said stable signal being processed to generate images.

11. Method according to claim 10, wherein said body is a living body.

12. Method according to claim 11, wherein said contrast medium or agent is injected into a blood vessel of said living body.

13. Method according to claim 10, wherein said excitation signal exerts a pressure in the range from 40 to 900 kPa, preferably from 60 to 500 kPa, and even more preferably from 60 to 200 kPa on said microbubbles.

14. Method according to claim 10, wherein said excitation signal is a sinusoidal signal.

15. Method according to claim 11, wherein said excitation signal is a sinusoidal signal.

16. Method according to claim 12, wherein said excitation signal is a sinusoidal signal.

17. Method according to claim 13, wherein said excitation signal is a sinusoidal signal.

18. Method according to claim 10, wherein each of said microbubbles consists of a membrane containing a gaseous medium.

19. Method according to claim 13, wherein each of said microbubbles consists of a membrane containing a gaseous medium.

20. Method according to claim 14, wherein each of said microbubbles consists of a membrane containing a gaseous medium.

21. Method according to claim 10, wherein a plurality of images obtained at successive instants of time of the echographic signal, or at spatially distinct points of said part under examination, are displayed simultaneously on a screen.

22. Ultrasonic method for imaging, in which an echographic contrast medium including microbubbles, or generating microbubbles upon exposure to ultrasonic waves, is introduced into a portion of a body under investigation and is struck by an ultrasonic excitation signal at an excitation frequency (f₀), and in which the microbubbles struck by the ultrasonic excitation signal generate an echo signal at a frequency different from the excitation frequency, said signal being used to generate an image, wherein said excitation signal exerts sufficient pressure on said microbubbles to cause their rupture, an echographic signal being generated during the rupture said signal containing a spectral distribution at the excitation frequency, at its subharmonics and at its ultraharmonics, said signal being filtered to extract the spectral content from it at at least two of said ultraharmonics and subharmonics.

23. Ultrasonic imaging method, including the steps of:
introducing a contrast medium including microbubbles, or generating microbubbles upon exposure to ultrasonic waves, in a portion under investigation of a body;
strucking said portion with an ultrasound excitation signal at an excitation signal, said microbubbles generating an echo signal at a frequency different from the excitation frequency;
wherein said excitation signal is controlled to exert a pressure on said microbubbles such that the microbubbles emit a stable signal at at least one subharmonic of said excitation frequency.

24. Method according to claim 23, wherein said excitation signal is controlled to exert a pressure between 30 kPa and 1 Mpa on said microbubbles.

25. Method according to claim 23, wherein said excitation signal exerts a pressure in the range from 40 to 900 kPa, preferably from 60 to 500 kPa, and even more preferably from 60 to 200 kPa on said microbubbles.

26. Ultrasonic imaging method, including the steps of:
injecting a contrast medium including microbubbles, or generating microbubbles upon exposure to ultrasonic waves, in a blood vessel of a patient;
strucking said microbubbles with an ultrasound excitation signal at an excitation signal, said microbubbles generating an echo signal at a frequency different from the excitation frequency; wherein said excitation signal is controlled to exert a pressure on said microbubbles such that the microbubbles emit a stable signal at at least one subharmonic of said excitation frequency.

27. Method according to claim 26, wherein said excitation signal is controlled to exert a pressure between 30 kPa and 1 Mpa on said microbubbles.

28. Method according to claim 26, wherein said excitation signal exerts a pressure in the range from 40 to 900 kPa, preferably from 60 to 500 kPa, and even more preferably from 60 to 200 kPa on said microbubbles.

29. Ultrasonic imaging method, including the steps of:
introducing a contrast medium including microbubbles, or generating microbubbles upon exposure to ultrasonic waves, in a portion under investigation of a body;
strucking said portion with an ultrasound excitation signal at an excitation signal, said microbubbles generating an echo signal at a frequency different from the excitation frequency;
wherein said excitation signal exerts sufficient pressure on said microbubbles to cause their rupture, an echographic signal being generated during the rupture said signal containing a spectral distribution at the excitation frequency, at its subharmonics and at its ultraharmonics, said signal being filtered to extract the spectral content from it at at least two of said ultraharmonics and subharmonics.

30. Ultrasonic imaging method, including the steps of:
injecting a contrast medium including microbubbles, or generating microbubbles upon exposure to ultrasonic waves, in a blood vessel of a patient;
strucking said microbubbles with an ultrasound excitation signal at an excitation signal, said microbubbles generating an echo signal at a frequency different from the excitation frequency;
wherein said excitation signal exerts sufficient pressure on said microbubbles to cause their rupture, an echographic signal being generated during the rupture said signal containing a spectral distribution at the excitation frequency, at its subharmonics and at its ultraharmonics, said signal being filtered to extract the spectral content from it at at least two of said ultraharmonics and subharmonics.

31. An ultrasonic imaging system for imaging the harmonic response of a structure inside a body, including:
means for transmitting ultrasonic energy into the body at an excitation frequency;
means responsive to said transmitted ultrasonic energy, for receiving ultrasonic echo signals, generated by microbubbles of a contrast medium introduced into said body, at a subharmonic of said excitation frequency;
means for producing an ultrasonic image from said echo signals;
wherein said excitation signal is controlled to exert a pressure on said microbubbles, so that the microbubbles emit a stable signal at one subharmonic at least of the excitation frequency, said stable signal being processed to generate images.

32. System according to claim 31, wherein said excitation signal is controlled to exert on said microbubbles a pressure between 30 kPa and 1 MPa, and preferably between 40 to 900 kPa, and more preferably from 60 to 500 kPa, and even more preferably from 60 to 200 kPa.

33. An ultrasonic imaging system for imaging the harmonic response of a structure inside a body, including:
means for transmitting ultrasonic energy into the body at an excitation frequency;
means responsive to said transmitted ultrasonic energy, for receiving ultrasonic echo signals, generated by microbubbles of a contrast medium introduced into said body, at a subharmonic of said excitation frequency;
means for producing an ultrasonic image from said echo signals;
wherein said excitation signal exerts sufficient pressure on said microbubbles to cause their rupture, an echographic signal being generated during the rupture said signal containing a spectral distribution at the excitation frequency, at its subharmonics and at its ultraharmonics, said means responsive to said transmitted ultrasonic energy including a filter to extract the spectral content from it at at least two of said ultraharmonics and subharmonics.
